(19) United States
(12) Patent Application Publication
(10) Pub. No.: US 2008/0165017 A1
(43) Pub. Date: Jul. 10, 2008

(54) Ear-mounted biosensor

(75) Inventor: Boris Schwartz, Hod Hasharon (IL)

Correspondence Address: DARBY & DARBY P.C., P.O. BOX 770, Church Street Station, New York, NY, 10008-0770, US

(73) Assignee: HIPPOC LTD., Hod Hasharon (IL)

(21) Appl. No.: 12/011,135

(22) Filed: Jan. 23, 2008

Related U.S. Application Data
(63) Continuation-in-part of application No. PCT/IL2006/000505, filed on Apr. 25, 2006.
(60) Provisional application No. 60/703,557, filed on Jul. 28, 2005.

(51) Int. Cl. A61B 5/1455

(52) U.S. Cl. 340/573.1; 600/324

(57) ABSTRACT

A physiological monitoring device includes a device housing shaped to fit behind an ear of a subject and a sensor attached to the device housing so as to sense a physiological characteristic of the subject at a location behind the ear. An earphone speaker is directed towards an ear canal of the subject and provides an audible communication to the subject responsively to the physiological characteristic.

### CROSS-REFERENCE TO RELATED APPLICATION

This application is a continuation-in-part of PCT Patent Application PCT/IL2006/000505, filed Apr. 25, 2006, which claims the benefit of U.S. Provisional Patent Application 60/703,557, filed on Jul. 28, 2005. Both of these related applications are incorporated herein by reference.

### FIELD OF THE INVENTION

The present invention relates generally to health care and specifically to methods and systems for monitoring subject well-being.

### BACKGROUND OF THE INVENTION

Two known indicators of physical and psychological stress are Galvanic Skin Response (GSR) and heart rate.

GSR (also known as electrodermal response, skin conductance response, or skin conductance level) is a measure of electrical conductivity of a subject's skin. GSR may be determined by applying a small voltage between two electrodes affixed to the skin and measuring the generated current. Often, GSR is measured at the tip of a subject's finger or on the palm of a hand. An example of a GSR sensor used in clinical settings is the Model V71-23 Isolated Skin Conductance Coupler, distributed by Coulbourne Instruments of Allentown, Pa.

Heart rate may be determined by photoplethysmography (PPG), which can also be used to measure variations in blood oxygen levels by pulse oximetry. Oximetry readings are generally made in terms of a percent of blood oxygen saturation (SpO₂). A PPG probe measures light transmitted through or reflected from arterial blood. In transmission PPG, light is generally transmitted through a thin appendage of the body. U.S. Pat. No. 4,301,808 to Taus, for example, whose disclosure is incorporated herein by reference, describes the use of transmission PPG to measure the pulse rate of a subject during physical exercise. Taus states that PPG readings be made through an appendage such as the ear, the nose septum, or the web between the forefinger and the thumb.

Reflective pulse oximetry measures light reflected from arteries beneath the surface of the skin. U.S. Pat. No. 6,553,242 to Sarussi, whose disclosure is incorporated herein by reference, describes the use of reflective pulse oximetry to measure heart rate, as well as indications of apnea in sleeping infants. Sarussi identifies several means of affixing an oximetry sensor to a subject's body, including a wristband, an ankle band, a sock, and a headband for making measurements at the subject's forehead.

U.S. Pat. No. 6,783,501 to Takahashi et al., whose disclosure is incorporated herein by reference, describes the use of pulse oximetry to measure heart rate from various locations on the head during exercise. Measurement locations described by Takahashi include the forehead and the ear canal. Heart rate feedback to the exerciser may be provided by an audio indication, which may be provided through an earphone, or by a visual indication, which may be provided on a screen attached to glasses worn by the exerciser.

U.S. Pat. No. 6,760,610 to Tschupp et al., whose disclosure is incorporated herein by reference, describes the use of pulse oximetry to measure blood oxygenation in combination with a measurement of blood carbon dioxide levels.

U.S. Patent Publication 2005/0033131 to Chen et al., whose disclosure is incorporated herein by reference, describes an ear sensor assembly that supports an oximetry sensor in the ear concha, using an extension that clips onto the ear lobe.

Wearable medical devices that monitor an individual's well-being are available on the market. For example, the SenseWear® Armband, distributed by Bodymedia of Pittsburgh, Pa., employs an accelerometer that records body movement, a temperature sensor that detects changes in skin temperature, and a GSR sensor that measures level of exertion during exercise.

Psychological stress among employees can have a significant impact on their job effectiveness and can lead to accidents, absenteeism, and employee turnover. According to an article by the American Institute of Stress, available at www.stress.org/job.htm and whose disclosure is incorporated herein by reference, workplace stress increases business costs in the U.S. by approximately $300 billion per year. Workplace testing of employees for indications of well-being is known in the art. For example, U.S. Pat. No. 6,352,516 to Pozos, et al., whose disclosure is incorporated herein by reference, describes a method for monitoring employee fatigue by measuring the force of fingers striking a keyboard.

### SUMMARY OF THE INVENTION

Embodiments of the present invention provide apparatus and methods for monitoring one or more physiological parameters from a location behind the ear. A sensor mounted to an earphone and positioned behind the ear is configured to sense the physiological parameters in a convenient, comfortable, and non-obtrusive manner.

Photoplethysmography (PPG) of arterial blood either in the scalp behind the ear or in the ear itself may be used to determine heart rate and/or oxygen saturation. Galvanic Skin Response (GSR) measurements may also be made from the location behind the ear.

The physiological parameters may be used to determine stress and other health indicators while an individual being monitored is performing activities in a non-medical setting, such as activities related to work or leisure. These indicators may be provided to the individual and/or to a health care institution, such as a remotely based hospital. The earphone to which the sensor is mounted may be utilized to provide an indication of the sensed parameters, as well as to provide additional functions that enhance the convenience of use.

There is therefore provided, in accordance with an embodiment of the present invention, a physiological monitoring device, including:

a device housing shaped to fit behind an ear of a subject;

a sensor attached to the device housing so as to sense a physiological characteristic of the subject at a location behind the ear; and

an earphone speaker directed towards an ear canal of the subject and operative to provide an audible communication to the subject responsively to the physiological characteristic.

The location may be on at least one of a scalp of the subject and a pinna of the subject, and the sensor may be operative to sense the physiological characteristic on both the scalp and the pinna.

In some embodiments, the device includes a photoplethysmographic (PPG) probe, which is adapted to sense a characteristic of arterial blood flow. The characteristic of arterial blood flow may include blood volume pulse (BVP), heart rate, blood oxygen saturation (SpO₂), or respiration rate.

The device may additionally or alternatively include a Galvanic Skin Response (GSR) sensor operative to sense a characteristic of skin. The GSR sensor typically includes two electrodes, which are positioned so as to contact the skin.

In some embodiments, the device includes a control unit, which is housed in the device housing and is operative to calculate a level of stress of the subject responsively to the physiological characteristic.

The device may also include a transmitter, which is housed in the device housing and is operative to transmit to an external receiver a signal indicative of the physiological characteristic.

The earphone speaker may be operative to play at least one of music and work-related communications.

The earphone speaker may extend from the device housing behind the ear into an opening of the ear canal. Alternatively, the device housing may include a speaker housing, which is shaped to surround the ear and is held against the ear by a headset.

There is further provided, in accordance with an embodiment of the present invention, a system for monitoring physiological parameters, including:

a physiological monitoring device, including:
a device housing shaped to fit behind an ear of a subject;
a sensor attached to the device housing so as to sense a physiological characteristic of the subject at a location behind the ear;
an earphone speaker directed towards an ear canal of the subject and operative to provide an audible communication to the subject; and
a transmitter housed in the device housing and operative to transmit a signal indicative of the physiological characteristic; and

a receiving device, separate from the physiological monitoring device and operative to receive and process the signal.

In some embodiments, the receiving device is operative to transmit an indication of the physiological characteristic over a communication network to a monitoring center.

The receiving device may be operative to transmit an audio signal to be played by the earphone speaker.

In further embodiments, the indication of the physiological characteristic is an indicator of stress.

Additionally, the physiological monitoring device may be included in a communication headset used by the subject in work-related communications.

There is also provided, in accordance with an embodiment of the present invention, a method for monitoring physiological parameters including:

fitting a physiological monitoring device behind an ear of a subject in such a manner that a sensor attached to the device housing is positioned behind the ear;

sensing a physiological characteristic of the subject using the sensor at the location behind the ear; and

responsively to the physiological characteristic, providing an audible communication through an earphone speaker attached to the housing and directed towards an ear canal of the subject.

In disclosed embodiments, sensing the physiological characteristic includes sensing a characteristic of arterial blood flow using a photoplethysmographic (PPG) probe.

Additionally or alternatively, the sensor includes a Galvanic Skin Response (GSR) sensor, the GSR sensor includes two electrodes, and sensing the physiological characteristic includes applying a voltage between the two electrodes and measuring a current generated through the scalp.

In some embodiments, the method includes calculating a level of stress of the subject responsively to the physiological characteristic.

In further embodiments, the method includes transmitting a signal indicative of the physiological characteristic from the physiological monitoring device to an external receiving device. The transmission may be made over a communication network to a monitoring center.

In further disclosed embodiments, the method includes playing from the earphone speaker at least one of music and work-related communications.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a monitoring device positioned behind the ear, in accordance with an embodiment of the present invention;

FIG. 2 is a schematic side view of the monitoring device of FIG. 1, in accordance with an embodiment of the present invention;

FIG. 3 is a schematic, pictorial illustration of a system for monitoring physiological parameters, in accordance with an embodiment of the present invention; and

FIG. 4 is a schematic, pictorial illustration of a monitoring device, in accordance with another embodiment of the present invention.

### DETAILED DESCRIPTION OF EMBODIMENTS

In the embodiments of the present invention that are described below, one or more physiological parameters are measured from a location that is on the scalp behind the ear.

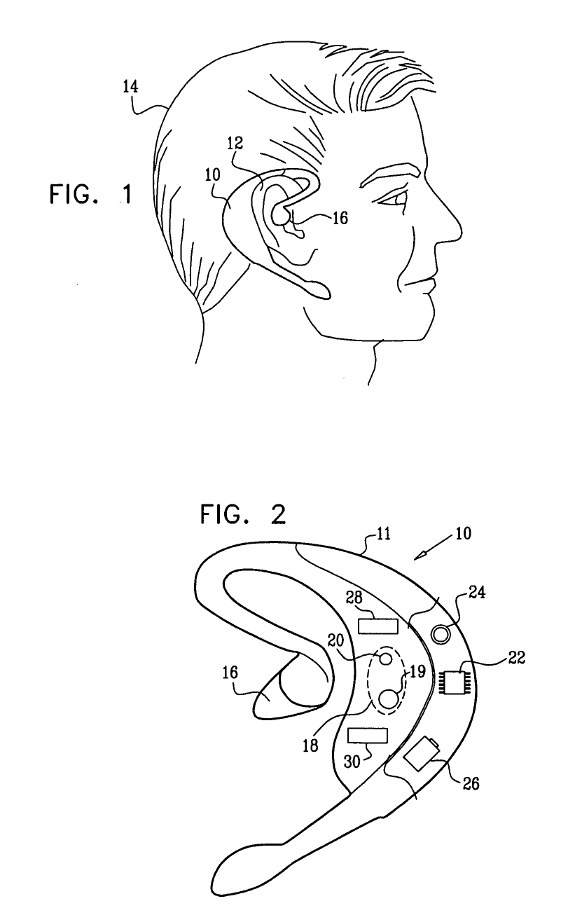

FIG. 1 is a schematic, pictorial illustration of a monitoring device 10 shaped to fit behind an ear 12 of a subject 14, in accordance with an embodiment of the present invention. The device fits between the scalp and the pinna, i.e., the cartilaginous portion of the external ear. Monitoring device 10 fits behind ear 12 in the manner of clip-on earphones known in the art so as to sense physiological parameters in a convenient, comfortable, and unobtrusive manner.

Sensors comprised in monitoring device 10 contact either a location on the scalp of subject 14 behind the ear 12 or a location on the back of the pinna, or both. The locations are chosen so as to overlie arteries beneath the skin, such as the occipital branch of the posterior auricular artery.

Monitoring device 10 comprises one or more photoplethysmographic (PPG) sensors, described further hereinbelow (FIG. 2), which are used to make oximetry measurements at the locations behind the ear. Additionally or alternatively, Galvanic Skin Response (GSR) measurements may be made behind the ear by a GSR sensor comprised in monitoring device 10 and described further hereinbelow.

Monitoring device 10 also comprises an earphone speaker 16 that extends from the monitoring device, in front of the ear, into the opening of the ear canal, thereby enabling subject 14 to receive an indication of the monitored parameters, as well as audio streams, such as music or work-related communications. Monitoring device 10 may be used while subject 14 is performing normal daily activities, such as work or leisure activities. When these activities require the use of an earphone, monitoring device 10 is particularly unobtrusive. For example, device 10 may be part of headset apparatus used by a customer service representative (CSR) in a call center environment.

FIG. 2 is a schematic side view of monitoring device 10, in accordance with an embodiment of the present invention. The monitoring device comprises a crescent-shaped housing 11 that fits between ear 12 and the scalp. For the sake of illustration, FIG. 2 shows the front side of housing 11, to which sensors are affixed. The back side of housing 11, not shown, may mirror the design of the front side and comprise similarly affixed sensors. Consequently, housing 11 may be placed behind either the left ear or the right ear of subject 14. Depending on the ear selected, one side of housing 11 is in contact with the scalp and the other side is in contact with the pinna. Alternatively, device 10 may be made with a sensor or sensors on only one side.

For the sake of illustration in the description that follows, the front side shown in FIG. 2 is assumed to be in contact with the subject's scalp. A PPG sensor 18 is affixed to the front side in such a manner that the sensor contacts the scalp. Sensor 18 comprises one or more light sources, such as a LED 19, and further comprises a light detector 20. The device housing is opaque, thereby preventing ambient light from reaching the location and interfering with the light generated by LED 19. The light generated by LED 19 is sensed by detector 20 after being reflected from arterial blood under the scalp, such as blood flow in the occipital branch of the posterior auricular artery. It is to be understood that this artery is noted by way of example and that another artery behind the ear may also be used for the PPG measurement.

A signal, indicative of the light reflected from the arterial blood, is transmitted from detector 20 to a control unit 22.

Control unit 22 processes the received signal in order to determine the subject's heart rate, as well as SpO₂ variation of arterial blood over time. Based on the received signal, control unit 22 may also determine the subject's respiratory rate, as described, for example, by Leonard et al., in “Standard Pulse Oximeters Can Be Used to Monitor Respiratory Rate,” Emergency Medicine Journal 20, pages 524-525 (2003), which is incorporated herein by reference. Additionally or alternatively, the control unit may determine the blood volume pulse (BVP).

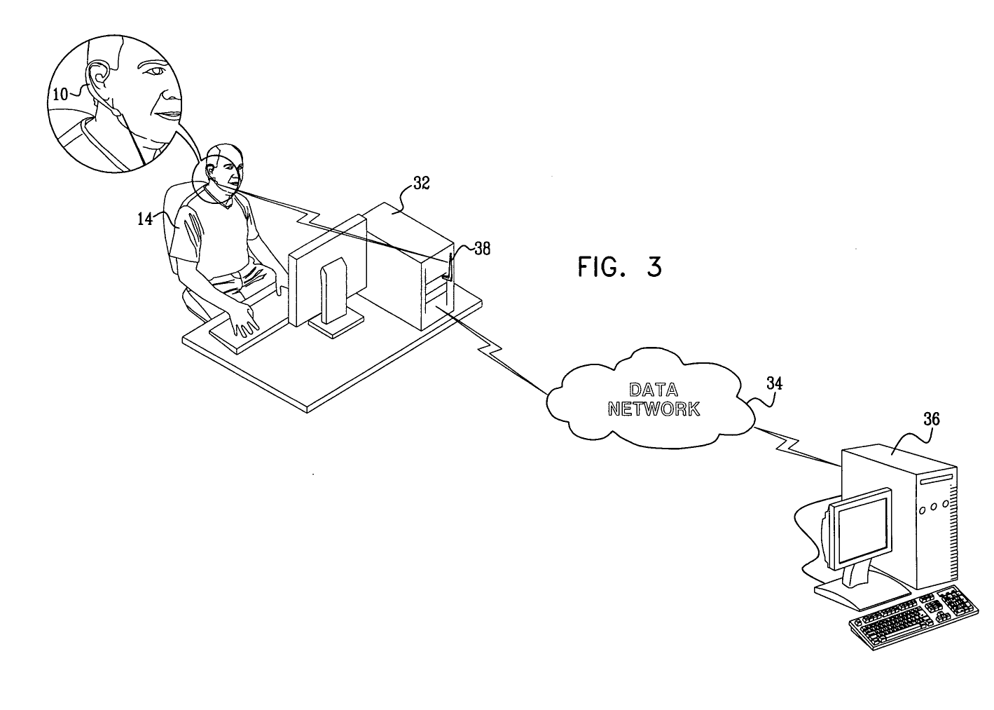

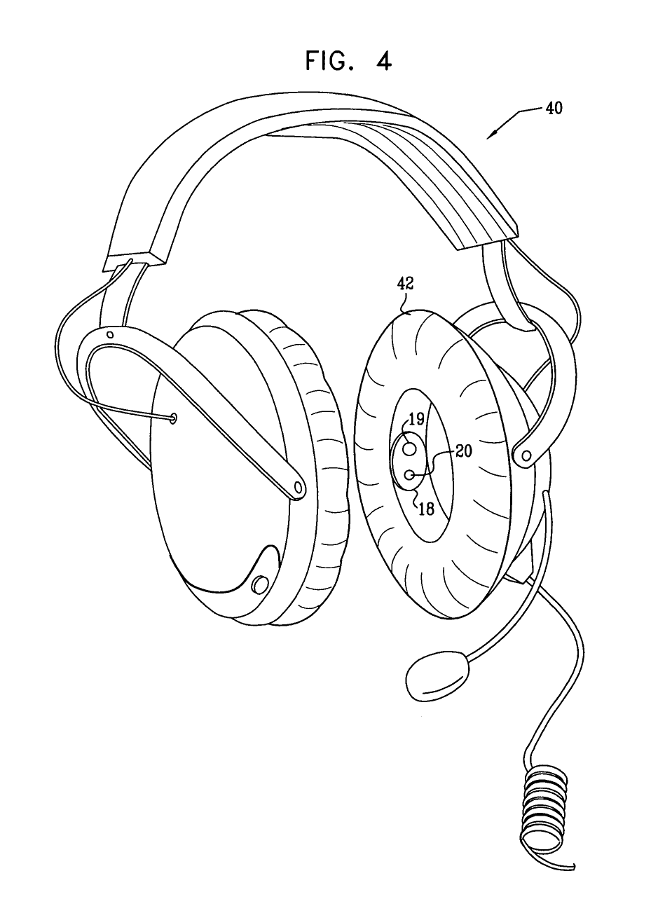

Control unit 22 may provide an audible indication of one or more of the determined physiological parameters, including heart rate, respiratory rate, or SpO₂ level to subject 14 via speaker 16. The indication may, for example, be in the form of a synthesized speech signal or an alarm in case the value of a monitored parameter is outside a predetermined range. Alternatively or additionally, the control unit transmits a signal indicative of one or more of the determined physiological parameters to an external receiver described hereinbelow (FIG. 3). To transmit the signal, control unit 22 may utilize a transmitter 24, which may transmit by Bluetooth™ wireless protocols, or by any other wireless or wired means known in the art. Power for LED 19, detector 20, control unit 22, and transmitter 24 is provided by a battery 26. Control unit 22 and battery 26 are typically comprised within the housing of monitoring device 10 and are therefore shown in the illustration within a cut-away portion of the device.

Additionally or alternatively, a GSR sensor, comprising a first electrode 28 and a second electrode 30, is also affixed to one or both sides of housing 11 so as to contact the skin. Respective electrodes 28 and 30 may be made of a conductive polymer, for example, thereby providing a good electrical contact with the scalp when the monitoring device is in place behind the ear. Control unit 22 passes a current between electrodes 28 and 30 in order to measure skin conductance between the electrodes. As in the case of the heart rate and SpO₂ measurements mentioned above, control unit 22 may process the GSR sensor signal in order to determine a level of stress and/or exertion and may give the subject an audible indication of the level via speaker 16. Alternatively or additionally, the control unit transmits a signal indicative of the skin conductance to an external receiver described hereinbelow (FIG. 3). To transmit the signal, control unit 22 may utilize transmitter 24.

In some embodiments of the present invention, the PPG and GSR measurements described above may be taken at the back of the pinna of ear 12 by sensors on the back side of housing 11 (not shown), instead of or in addition to the measurements made on the scalp. Measurements of physiological parameters at both the scalp and the back of the pinna may be made simultaneously by respective sensors on each of the front and back sides of the housing. Circuitry in the housing, such as control unit 22, may be configured to determine which of the scalp and ear locations provides a better signal-to-noise ratio (SNR). The parameters measured at the location with the better SNR may then be selected for further processing and transmission, as described below. Alternatively, the measurements may be averaged, or other selection criteria may be applied.

FIG. 3 is a schematic, pictorial illustration of a system for monitoring physiological parameters, in accordance with an embodiment of the present invention. While subject 14 has device 10 in place behind his ear, he may perform normal daily activities, including activities related to his work or leisure.

PPG and skin conductance data transmitted from monitoring device 10 may be used to determine a level of subject stress and changes in that level. Indicators of stress are, for example, increased heart rate, increased respiratory rate, and increased skin conductance. To report stress level, monitoring device 10 may transmit physiological data to a receiving device such as a cell phone, or a personal computer (PC) 32. PC 32 is configured to receive the signal transmitted by transmitter 24 by wireless or wired means. When wireless means, such as Bluetooth transmission, are utilized, PC 32 may receive such transmission by means of an antenna 38. The PC may also return an audio signal to be played through earphone speaker 16.

The calculation of stress level from physiological parameters may be determined by device 10 or by PC 32. The PC may be configured to display a stress level to the subject. Alternatively, or additionally, PC 32, or another receiving device, such as a cell phone, may be configured to transmit physiological parameters over a data network 34, to a monitoring center 36, which may be maintained by a health care provider or by the subject's employer, for example. The monitoring center may be programmed to automatically notify the subject and other concerned parties, such as the subject's doctor or work supervisor, if changes in the level of stress, or changes in other physiological indicators, warrant intervention.

FIG. 4 is a schematic, pictorial illustration of a monitoring device 40, in accordance with another embodiment of the present invention. In this case, device 40 comprises a headset, which holds speaker housings 42 against the subject's ears. Speaker housing 42 surrounds and thus fits behind the subject's ear. Sensor 18 is mounted inside one of the speaker housings, as shown in the figure, so as to fit behind the ear.

Although the embodiments described above relate specifically to the measurement of heart rate, SpO₂, respiratory rate, and skin conductance, the principles of the present invention may also be applied to other types of measurements indicative of subject well-being or stress. Furthermore, although these embodiments make reference to certain types of active life settings and signaling methods, the principles of the present invention may likewise be applied in the context of other environments and other communications technologies.

It will thus be appreciated that embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A physiological monitoring device, comprising:
a device housing shaped to fit behind an ear of a subject;
a sensor attached to the device housing so as to sense a physiological characteristic of the subject at a location behind the ear; and
an earphone speaker directed towards an ear canal of the subject and operative to provide an audible communication to the subject responsively to the physiological characteristic.

2. The device of claim 1, wherein the location is on at least one of a scalp of the subject and a pinna of the subject.

3. The device of claim 2, wherein the sensor is operative to sense the physiological characteristic on both the scalp and the pinna of the subject.

4. The device of claim 1, wherein the sensor comprises a photoplethysmographic (PPG) probe, which is adapted to sense a characteristic of arterial blood flow.

5. The device of claim 4, wherein the characteristic of arterial blood flow comprises at least one of blood volume pulse (BVP), heart rate, blood oxygen saturation (SpO₂), and respiration rate.

6. The device of claim 1, wherein the sensor comprises a Galvanic Skin Response (GSR) sensor operative to sense a characteristic of skin.

7. The device of claim 6, wherein the GSR sensor comprises two electrodes, which are positioned so as to contact the skin.

8. The device of claim 1, and comprising a control unit, which is housed in the device housing and is operative to calculate a level of stress of the subject responsively to the physiological characteristic.

9. The device of claim 1, and comprising a transmitter, which is housed in the device housing and is operative to transmit to an external receiver a signal indicative of the physiological characteristic.

10. The device of claim 1, wherein the earphone speaker is operative to play at least one of music and work-related communications.

11. The device of claim 1, wherein the earphone speaker extends from the device housing behind the ear into an opening of the ear canal.

12. The device of claim 1, wherein the device housing comprises a speaker housing, which is shaped to surround the ear and is held against the ear by a headset.

13. A system for monitoring physiological parameters, comprising:
a physiological monitoring device, comprising:
a device housing shaped to fit behind an ear of a subject;
a sensor attached to the device housing so as to sense a physiological characteristic of the subject at a location behind the ear;
an earphone speaker directed towards an ear canal of the subject and operative to provide an audible communication to the subject; and
a transmitter housed in the device housing and operative to transmit a signal indicative of the physiological characteristic; and
a receiving device, separate from the physiological monitoring device and operative to receive and process the signal.

14. The system of claim 13, wherein the receiving device is operative to transmit an indication of the physiological characteristic over a communication network to a monitoring center.

15. The system of claim 13, wherein the receiving device is operative to transmit an audio signal to be played by the earphone speaker.

16. The system of claim 13, wherein the indication of the physiological characteristic is an indicator of stress.

17. The system of claim 13, wherein the physiological monitoring device is comprised in a communication headset used by the subject in work-related communications.

18. A method for monitoring physiological parameters comprising:
fitting a physiological monitoring device behind an ear of a subject in such a manner that a sensor attached to the device housing is positioned at a location behind the ear of the subject;
sensing a physiological characteristic using the sensor on the location; and
responsively to the physiological characteristic, providing an audible communication through an earphone speaker attached to the housing and directed towards an ear canal of the subject.

19. The method of claim 18, wherein the location is on at least one of a scalp of the subject and a pinna of the subject.

20. The method of claim 19, wherein the sensor is operative to sense the physiological characteristic on both the scalp and the pinna of the subject.

21. The method according to claim 18, wherein the sensor comprises a photoplethysmographic (PPG) probe, and wherein sensing the physiological characteristic comprises sensing a characteristic of arterial blood flow using the PPG probe.

22. The method of claim 18, wherein the sensor comprises a Galvanic Skin Response (GSR) sensor, wherein the GSR sensor comprises two electrodes, and wherein sensing the physiological characteristic comprises applying a voltage between the two electrodes and measuring a current generated through the scalp.

23. The method of claim 18, and comprising calculating a level of stress of the subject responsively to the physiological characteristic.

24. The method of claim 18, and comprising transmitting a signal indicative of the physiological characteristic from the physiological monitoring device to an external receiving device.

25. The method of claim 24, and comprising transmitting an indication of the physiological characteristic from the receiving device over a communication network to a monitoring center.

26. The method of claim 18, and comprising playing from the earphone speaker at least one of music and work-related communications.
